(19) United States
(12) Patent Application Publication
(10) Pub. No.: US 2017/0340381 A1
(43) Pub. Date: Nov. 30, 2017

(54) SURGICAL INSTRUMENT INCORPORATING A CIRCUIT BOARD AND METHODS OF MANUFACTURING THE SAME

(71) Applicant: COVIDIEN LP, Mansfield, MA (US)

(72) Inventor: JAMES D. ALLEN, IV, BROOMFIELD, CO (US)

(21) Appl. No.: 15/593,474

(22) Filed: May 12, 2017

Related U.S. Application Data
(60) Provisional application No. 62/340,598, filed on May 24, 2016.

(51) Int. Cl. A61B 18/14; A61B 34/00; A61B 34/35; A61B 34/37; A61B 90/00; A61B 18/00; A61B 17/00

(52) U.S. Cl. CPC A61B 18/1445; A61B 34/37; A61B 34/74; A61B 34/76; A61B 90/06; A61B 34/35; A61B 2090/067; A61B 2017/00526; A61B 2018/00791; A61B 2018/00875; A61B 2018/1455; A61B 2090/064

(57) ABSTRACT

A surgical instrument includes a housing, a shaft, an end effector assembly, and a circuit board. The housing includes at least one first electrical connector adapted to connect to an energy source. The shaft extends from the housing. The end effector assembly is disposed proximate a distal end of the shaft and includes at least one second electrical connector. The circuit board is positioned on the shaft. The circuit board extends from a proximal end to the distal end of the shaft and includes at least one proximal contact at a proximal end thereof configured to electrically couple to the at least one first electrical connector and at least one distal contact at a distal end thereof configured to electrically couple to the at least one second electrical connector to thereby electrically couple the energy source to the end effector assembly.

### CROSS REFERENCE TO RELATED APPLICATION

The present application claims the benefit of and priority to U.S. Provisional Application Ser. No. 62/340,598, filed on May 24, 2016, the entire contents of which are incorporated herein by reference.

### BACKGROUND

### Technical Field

The present disclosure relates to surgical instruments and, more particularly, to a surgical forceps configured to treat and/or cut tissue utilizing one or more circuit boards, and methods of manufacturing the same.

### Background of Related Art

A surgical forceps is a plier-like device which relies on mechanical action between its jaws to grasp, clamp, and constrict tissue. Energy-based surgical forceps utilize both mechanical clamping action and energy to treat, e.g., coagulate, cauterize, and/or seal, tissue. Typically, once tissue is treated, the surgeon has to accurately sever the tissue. Accordingly, many devices have been designed which incorporate a knife or blade member which effectively severs tissue after treatment thereof.

As an alternative to open forceps for use with open surgical procedures, many modern surgeons use endoscopic or laparoscopic instruments for remotely accessing tissue through smaller, puncture-like incisions or natural orifices. As a direct result thereof, patients tend to benefit from less scarring and reduced healing time. Endoscopic instruments are typically inserted into the patient through a cannula, or port. Smaller cannulas are usually preferred, which, as can be appreciated, ultimately presents a design challenge to instrument manufacturers who must find ways to make endoscopic instruments that fit through the smaller cannulas without compromising functionality.

Currently, energy-based surgical forceps are designed to include one or more circuit boards, which are typically positioned within a housing of the energy-based surgical forceps.

### SUMMARY

In accordance with the present disclosure, a surgical instrument includes a housing, a shaft, an end effector assembly, and a circuit board. The housing includes at least one first electrical connector adapted to connect to an energy source and the shaft extends distally therefrom. The shaft including a proximal end coupled to the housing and a distal end spaced-apart from the housing. The end effector assembly is disposed proximate the distal end of the shaft and includes at least one second electrical connector. The circuit board is positioned on the shaft. The circuit board extends from the proximal end of the shaft to the distal end of the shaft and includes at least one proximal contact at a proximal end thereof configured to electrically couple to the at least one first electrical connector and at least one distal contact at a distal end thereof configured to electrically couple to the at least one second electrical connector to thereby electrically couple the energy source to the end effector assembly.

In an aspect of the present disclosure, the circuit board is a flexible circuit board positioned on an interior surface of the shaft. In another embodiment, the circuit board is formed on an interior surface of the shaft. In yet another embodiment, the circuit board is formed on an exterior surface of a tube and the tube is disposed within the shaft.

In another aspect of the present disclosure, the circuit board includes a sensing circuit capable of measuring impedance and tissue response during a sealing cycle. In another embodiment, the circuit board includes a feedback loop capable of measuring energy and stabilizing, increasing or decreasing a signal supplied by the energy source to the end effector assembly. In yet another embodiment, the circuit board includes a switch mechanism containing a multi-pole contact switch.

In another aspect of the present disclosure, the end effector assembly includes a first jaw member and a second jaw member. In an embodiment, the end effector assembly and the shaft each include a sensor and each sensor is electrically coupled to a feedback circuit located on the circuit board. The feedback circuit calculates an angle of the first jaw member and the second jaw member. In another embodiment, the circuit board includes a PZT sensor capable of measuring a jaw force of the first jaw member and the second jaw member.

In a method of manufacturing a surgical instrument, a circuit board is positioned on a shaft having a proximal end and a distal end such that the circuit board extends from the proximal end of the shaft to the distal end of the shaft, and at least one proximal contact of the circuit board is disposed at the proximal end of the shaft, and at least one distal contact of the circuit board disposed at the distal end of the shaft. Also, the at least one proximal contact of the circuit board is connected to at least one first electrical connector and the at least one distal contact of the circuit board is connected to at least second electrical connector. The proximal end of the shaft is connected to a housing and the distal end of the shaft is connected to an end effector assembly. The circuit board may be formed via laser etching, physical etching, chemical etching or depositing.

In one aspect of the present disclosure, a method for manufacturing a surgical instrument includes positioning a circuit board within a shaft by aligning the at least one proximal contact with at least one proximal contact aperture defined through the shaft and aligning the at least one distal contact with at least one distal contact aperture defined through the shaft.

In another aspect of the present disclosure, a method for manufacturing a surgical instrument includes positioning a circuit board on a shaft including a flexible circuit board positioned to an interior surface of the shaft. Positioning the flexible circuit board to the interior surface of the shaft includes providing a flexible material; forming a circuit board upon the flexible material; positioning a proximal end of the circuit board to a proximal end of the shaft and a distal end of the circuit board to a distal end of the shaft; and positioning the circuit board on the interior surface of the shaft. In some embodiments, positioning the circuit board to the interior surface of the shaft may include a soldering method.

In still another aspect of the present disclosure, a method for manufacturing a surgical instrument includes connecting an electrical wire to the shaft by connecting the shaft to a distal end of the housing, connecting the electrical wire to a proximal end of the shaft, and connecting the end effector assembly to a distal end of the shaft. Electrosurgical energy is provided to the electrical wire.

In a method of manufacturing a shaft of a surgical instrument, the method includes providing a flexible substrate and a circuit board. The circuit board is positioned on the flexible substrate, and has at least one proximal contact and at least on distal contact. The flexible substrate is formed into a tubular shaft having the circuit board on an interior surface thereof with the at least one proximal contact disposed at a proximal end of the shaft and the at least one distal contact disposed at a distal end of the shaft. The proximal end of the shaft is connected to a housing, connecting at least one first electrical connector housed within the housing to the at least one proximal contact and the distal end of the shaft is connected to an end effector assembly, connecting at least one second electrical connector of the end effector assembly to the at least one distal contact.

In another method of manufacturing a shaft of a surgical instrument, the method includes forming a tube. The tube includes a body defining an inner surface and an outer surface, and a central passageway extending longitudinally through the body. While forming the tube, a plurality of electrical wires is positioned through the outer surface of the body such that the plurality of electrical wires extends from a proximal end of the tube to a distal end of the tube. A portion of at least one of the plurality of electrical wires is exposed. A housing is connected to the proximal end of the tube and an end effector assembly is connected to the distal end of the tube.

### BRIEF DESCRIPTION OF THE DRAWINGS

Various aspects and features of the present disclosure are described herein with reference to the drawings wherein:

FIG. 1 is a front, perspective view of an surgical instrument provided in accordance with the present disclosure;

FIG. 2 is a side, cut-away view of a proximal portion of the surgical instrument of FIG. 1, wherein a housing and some of the internal components thereof have been removed;

FIG. 3 is an enlarge, exploded view of a circuit board positionable within a tube placed within a shaft of the surgical instrument of FIG. 1;

FIGS. 4A and 4B are respective side and perspective views of the circuit board formed on an interior surface of the shaft;

FIG. 5 is an enlarged, exploded view of the flexible circuit board positionable within the interior surface of the shaft;

FIG. 6 is a perspective view of the shaft including a plurality of electrical wires;

FIG. 7 is a front, perspective view of an end effector assembly of the surgical instrument of FIG. 1, disposed in a spaced-apart position;

FIG. 8 is a front, perspective view of the end effector assembly of the surgical instrument of FIG. 1, disposed in an approximated position; and

FIG. 9 is a schematic of a robotic surgical system configured to work with surgical instrument of this present disclosure.

### DETAILED DESCRIPTION

Embodiments of the present disclosure are now disclosed in detail with reference to the drawings in which like reference numerals designate or corresponding elements in each of the several views. Throughout this description, the term “proximal” will refer to the portion of the device or component thereof that is closer to the clinician and the term “distal” will refer to the portion of the device or component thereof that is farthest from the clinician.

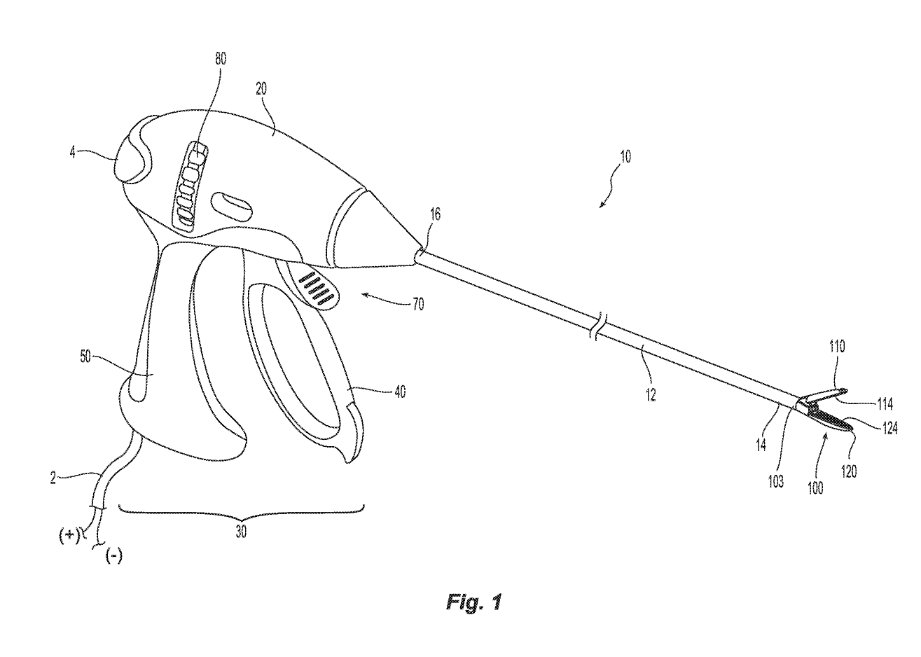

Turning to FIG. 1, a handheld, shaft-based, or endoscopic surgical forcep 10 is depicted. For the purpose herein, forceps 10 or any other suitable surgical instrument may be utilized in accordance with the present disclosure. Obviously, different electrical and mechanical connections and considerations apply to each particular type of instrument; however, the aspects and features of the present disclosure remain generally consistent regardless of the particular instrument used.

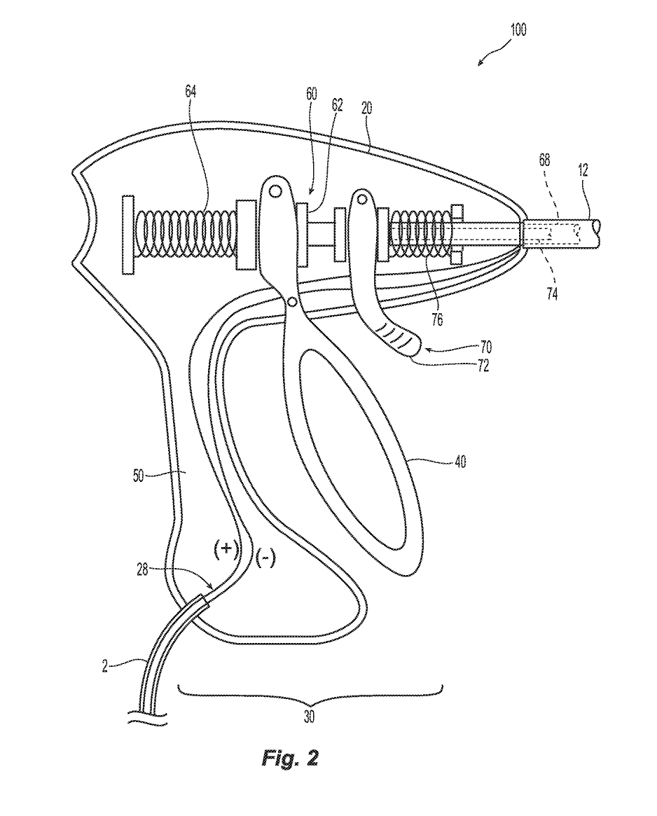

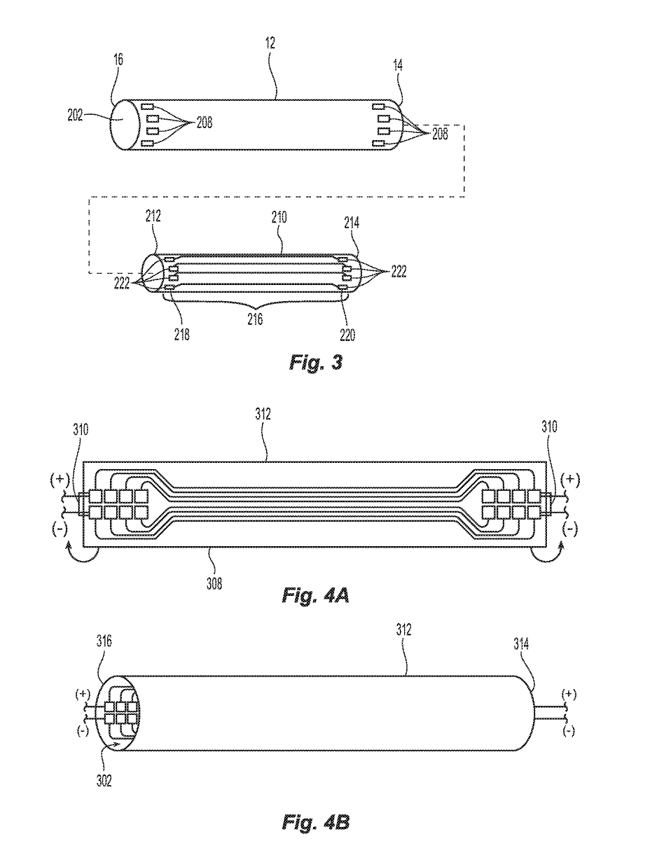

Referring to FIG. 1, surgical forceps 10 generally includes a housing 20, a handle assembly 30, a trigger assembly 70, a rotating assembly 80, an activation switch 4, and an end effector assembly 100. Forceps 10 further includes a shaft 12 having a distal end 14 configured to mechanically engage end effector assembly 100 and a proximal end 16 to mechanically engage housing 20. Forceps 10 also includes cable 2 that connects the forceps 10 to an electrosurgical energy source (not shown), e.g., a generator or other suitable power source, although forceps 10 may alternatively be configured as a battery-powered device. Cable 2 includes an electrical wire (or electrical wires) 28 (See FIG. 2) extending therethrough that has sufficient length to extend through housing 20 and operably couple to the shaft 12 or other suitable electrical contacts 222, 310, and 424 (FIGS. 3-6) in order to provide energy to the end effector assembly 100 from the energy source for selectively treating, e.g., cauterizing, coagulating/desiccating, and/or sealing, tissue.

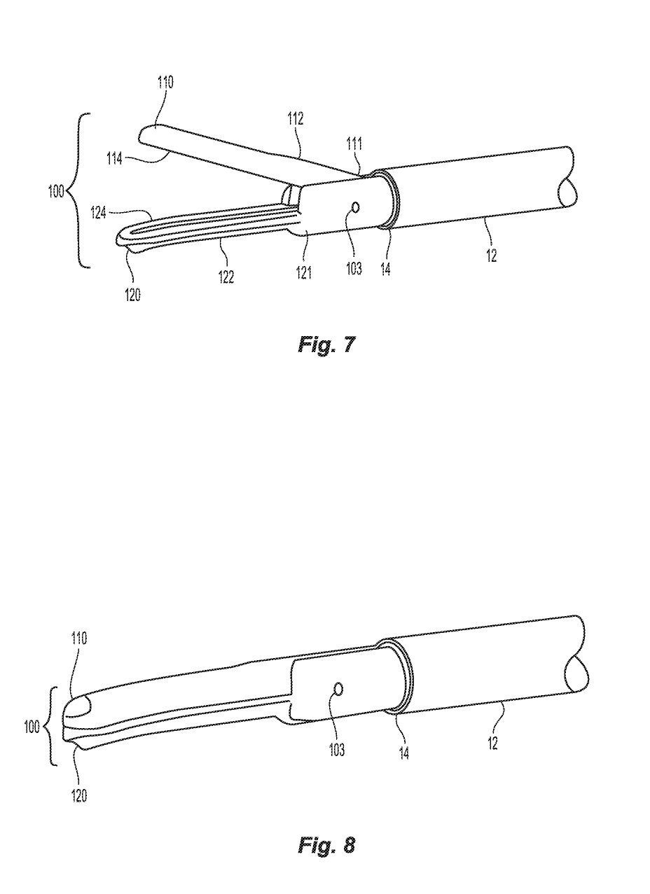

Referring to FIGS. 1 and 2, handle assembly 30 includes fixed handle 50 and a movable handle 40. Fixed handle 50 is integrally associated with housing 20 and handle 40 is movable relative to fixed handle 50. Movable handle 40 of handle assembly 30 is operably coupled to a drive assembly 60 that, together, mechanically cooperate to impart movement of jaw members 110, 120 of the end effector assembly 100 about a pivot 103 (FIGS. 7 and 8) between a spaced-apart position and an approximated position to grasp tissue between jaw members 110, 120. In particular, movable handle 40 is coupled to drive bar 68 via a drive mandrel 62 such that movement of movable handle 40 relative to housing 20 effects longitudinal translation of drive bar 68 through housing 20 and shaft 12. The distal end of drive bar 68 is coupled to one or both jaw members 110, 120 such that longitudinal translation of drive bar 68 pivots one or both of jaw members 110, 120 relative to the other. As shown in FIG. 1, movable handle 40 is initially spaced-apart from fixed handle 50 and, correspondingly, jaw members 110, 120 are disposed in the spaced-apart position (see also FIG. 7). Movable handle 40 is depressible from this initial position to a depressed position corresponding to the approximated position of jaw members 110, 120 (see FIG. 8). Further, a biasing member, e.g. spring 64, may be disposed within housing 20 and positioned to bias drive bar 68 distally, thereby biasing jaw members 110, 120 towards the spaced-apart position. However, other configurations are also contemplated.

With continued reference to FIGS. 1 and 2, trigger assembly 70 includes a trigger 72 coupled to housing 20 and movable relative thereto between an un-actuated position and an actuated position. More specifically, trigger 72 is operably coupled to an actuation bar 74 such that movement of trigger 72 relative to housing 20 effects longitudinal translation of actuation bar 74 through housing 20 and shaft 12. The distal end of actuation bar 74 is coupled to a knife (not shown) such that longitudinal translation of actuation bar 74 effects translation of the knife within the end effector assembly 100 from a retracted position relative to one or both jaw members 110, 120 to an extended position, wherein the knife extends between jaw members 110, 120 to cut tissue grasped therebetween. Trigger 72, as shown in FIG. 1, is initially disposed in an un-actuated position and, correspondingly, the knife is disposed in the retracted position. Trigger 72 is selectively actuatable from this un-actuated position to an actuated position corresponding to the extended position of the knife for cutting tissue grasped between jaw members 110, 120. Further, a biasing member, knife spring 76, is disposed within housing 20 and is positioned to bias actuation bar 74 proximally, thereby biasing the knife towards the retracted position and trigger 72 towards the un-actuated position.

Turning to FIG. 3, a shaft 12 of forceps 10 (FIG. 1) is shown. Shaft 12 includes a proximal end 16 and a distal end 14, both of which includes a plurality of electrical connectors 208. Shaft 12 further defines a lumen 202 therethrough. A tube 210 is configured to fit within lumen 202 of shaft 12. Tube 210 includes a proximal end 212 and a distal end 214. A circuit board 216 is positioned on an outer surface of tube 210. Circuit board 216 has a proximal end 218, a distal end 220, and a plurality of contacts 222 at each end. Circuit board 216 is positioned on tube 210 so that proximal end 218 of circuit board 216 aligns with proximal end 212 of tube 210 and distal end 220 of circuit board 216 aligns with distal end 214 of tube 210. The circuit board 216 extends the entire length of the tube 210; however, circuit board 216 may only partially extend across the length of the tube 210, depending upon a particular purpose.

Placement of tube 210 within shaft 12 aligns the plurality of electrical connectors 208 on shaft 12 with the plurality of contacts 222 of circuit board 216. The plurality of contacts 222 of circuit board 216, located adjacent the proximal end 212 and distal end 214 of tube 210, connect with the plurality of electrical connectors 208 of shaft 12, located on the proximal end 16 and distal end 14 of shaft 12. After placing tube 210 within shaft 12, the proximal end 16 of shaft 12 is connected to housing 20 (FIG. 1) and the distal end 14 is connected to end effector assembly 100 (FIG. 1). As described above, the energy source or other suitable power source provide energy to forceps 10 via cable 2. As mentioned above, electrical wires 28 are configured to connect to the plurality of electrical connectors 208 and supply energy to the plurality of electrical connectors 208 and the plurality of contacts 222. Circuit board 216, in turn, transfers the supplied energy to the respective jaw members 110, 120 of the end effector assembly 100.

Shaft 12 may include a plurality of contact apertures (not explicitly shown), whereas the plurality of electrical connectors 208 are located within housing 20 and end effector assembly 100. In this embodiment, the plurality of contact apertures is located on the proximal end 16 and the distal end 14 of shaft 12. Placement of tube 210 within shaft 12 aligns the plurality of contact apertures with the plurality of contacts 222 located adjacent the proximal end 212 and distal end 214 of tube 210. After alignment of the plurality of contact apertures and the plurality of contacts 222, shaft 12 is connected to the housing 20 and the end effector assembly 100, allowing the plurality of electrical connectors 208 and the plurality of contacts 222 to connect.

Turning to FIGS. 4A and 4B, another embodiment of shaft 312 of forceps 10 (FIG. 1) is shown having a circuit board 308 and a lumen 302 defined therethrough. Circuit board 308 includes a plurality of contacts 310 placed adjacent to both a proximal end 316 and a distal end 314 of shaft 312. In this configuration of shaft 312, circuit board 308 is positioned on a suitable flexible substrate (FIG. 4A), prior to forming the generally tubular shape of shaft 312. After circuit board 308 is positioned on the surface of the flexible substrate, shaft 312 is shaped to form tubular shaft 312.

Shaft 312 is connected to housing 20 and end effector assembly 100, allowing the plurality of contacts 310, located at the proximal end 316 and distal end 314 of shaft 312, to connect with a plurality of electrical connectors (not explicitly shown) located in the housing 20 and end effector assembly 100. As described above, electrical wires 28 connect to electrical connectors located in housing 20, which transfer the electrosurgical energy to the plurality of contacts 310 located at proximal end 316 of shaft 312. Circuit board 308 transfers the electrosurgical energy to the plurality of contacts 310 located at distal end 314, which, in turn, supplies energy to the end effector assembly 100.

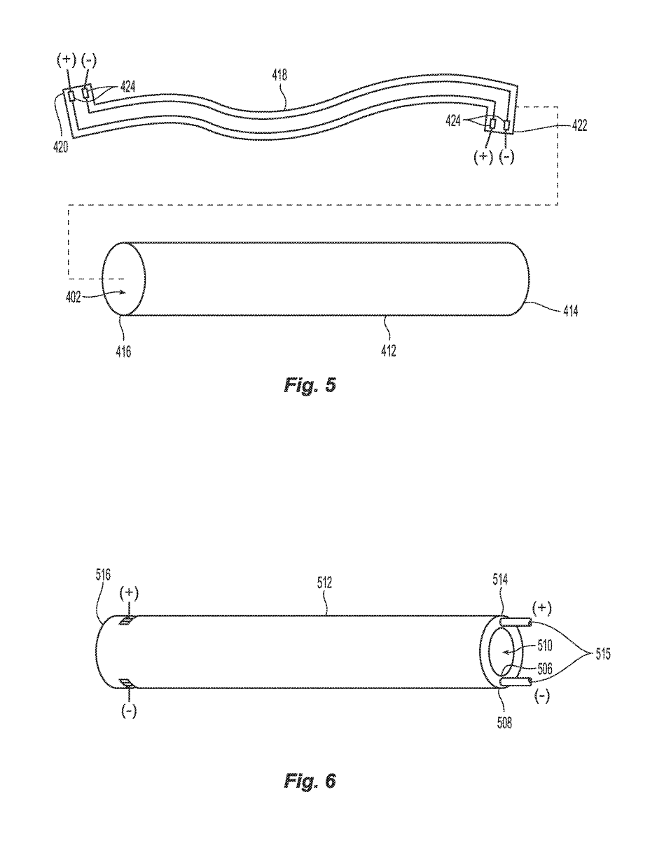

FIG. 5 shows another embodiment of a shaft 412 for use with forceps 10 (FIG. 1). Shaft 412 includes a proximal end 416, a distal end 414, a flexible circuit board 418 and lumen 402 defined therethrough. Flexible circuit board 418 has a proximal end 420, a distal end 422, and a plurality of contacts 424 located on both ends. Flexible circuit board 418 may include varying shapes, such as a rectangular shape, a wave-like shape, an “S” shape and/or an arcuate shape. Flexible circuit board 418 may be mounted on a flexible plastic substrate, such as polyimide, polyether ether ketone or any other suitable non-conductive polyester. Flexible circuit board 418 is positioned within lumen 402 and on an inner peripheral surface of shaft 412. Flexible circuit board 418 is positioned so that proximal end 420 of flexible circuit board 418 aligns with proximal end 416 of shaft 412 and distal end 422 of flexible circuit board 418 aligns with distal end 414 of shaft 412. Shaft 412 may also include a plurality of electrical connectors (not explicitly shown) located at both the proximal end 416 and the distal end 414 of shaft 412, which connect to the plurality of contacts 424 of flexible circuit board 418. The plurality of electrical connectors may be located in housing 20 and on end effector assembly 100 in a manner similar to the embodiments described above. In either instance, the plurality of electrical connectors allow flexible circuit board 418 to supply electrosurgical energy to the end effector assembly 100 after the proximal end 416 of shaft 412 is connected to housing 20 (FIG. 1) and the distal end 414 of shaft 412 is connected to end effector assembly 100 (FIG. 1). Electrosurgical energy is supplied to flexible circuit board 418, as described above, which, in turn, provides the supplied electrosurgical energy to end effector assembly 100, as described above.

Each circuit board described above may be positioned on the appropriate surface by a soldering method or any other known appropriate method. Additionally, each circuit board may be formed via laser etching, physical etching, chemical etching and/or depositing. Also, each electrical connector described above may be a ring connector and the plurality of contacts of each circuit board described above may be brush contacts.

In some embodiments, circuit boards 216, 308, and 418 include a sensing circuit (not specific shown) capable of measuring impedance and tissue response. The sensing circuit measures tissue response during the sealing cycle to provide information regarding the completion and accuracy of the sealing cycle. During the sealing cycle tissue responds to the applied electrosurgical energy by losing moisture and shrinking. The sensing circuit measures the tissue impedance to gauge the progression of the sealing cycle. The sensing circuit also measures the impedance of the electrosurgical energy being supplied by the energy source. Examples of sensing circuits are shown and described in commonly owned U.S. Pat. No. 8,216,223 entitled “SYSTEM AND METHOD FOR TISSUE SEALING” which is hereby incorporated by reference herein in its entirety.

In additional embodiments, circuit boards 216, 308, and 418 include a feedback loop circuit (not specifically shown) capable of measuring an applied and/or reflected energy and using that measurement to stabilize, increase or decrease the signal supplied by the energy source to the end effector assembly 100.

Circuit boards 216, 308, and 418 include a switch mechanism (not specifically shown) which contains a multi-pole contact switch having a low-level activation line, or contacts, which is contacted or connected after the contact or connection for the high-energy source is made, to activate the energy source. The reverse order of operation will deactivate the high-energy source before the high-energy contacts are separated. By operating the high-energy contacts and low-level activation line or contacts in this order, arcing associated with direct switching of high-energy source and the negative effects associated therewith are avoided. Examples of switch mechanism are shown and described in commonly owned U.S. Pat. No. 7,837,685 entitled “SWITCH MECHANISMS FOR SAFE ACTIVATION ON ENERGY ON AN ELECTROSURGICAL INSTRUMENT” which is hereby incorporated by reference herein in its entirety.

One of the jaw member 110, 120 of end effector assembly 100 includes a sensor (not specifically shown) and all embodiments of the shaft include a corresponding sensor (not specifically shown), which are electrically coupled to a feedback circuit located on circuit boards 216, 308, and 418 associated with the energy source or an independent controller. In use, as jaw members 110, 120 move proximate to one another, end effector assembly 100 sensor moves proximate to shaft sensor. Circuit boards 216, 308, and 418 feedback circuit calculates an angle of jaw members 110, 120 and provides signal back to the generator, which in turn provides information to a clinician. Examples of measuring the jaw members' angles at the onset of the sealing cycle and after the sealing cycle are shown and described in commonly owned U.S. Pat. No. 8,357,158 entitled “JAW CLOSURE DETECTION SYSTEM” which is hereby incorporated by reference herein in its entirety.

The forceps 10 includes a piezoelectric (PZT) component, such as a PZT sensor (not specific shown). The PZT sensor has the capability of sensing mechanical energy of jaw members 110, 120 and converting mechanical energy of jaw members 110, 120 to an electrical charge. Circuit boards 216, 308, and 418 receive the electrical charge generated by the PZT sensor and measure the jaw force of jaw members 110, 120. The circuit boards 216, 308, and 418 may also measure the stress, strain, and pressure of the jaw members 110, 120.

FIG. 6 shows another embodiment of a shaft 512 for use with forceps 10 (FIG. 1). Shaft 512 includes a proximal end 516, a distal end 514, an inner surface 506, an outer surface 508, a lumen 510 defined therethrough, and a plurality of electrical wires 515 each of the plurality of electrical wires 515 includes a proximal end and a distal end. While shaft 512 is being formed, the plurality of electrical wires 515 is positioned between outer surface 508 and inner surface 506, so that the plurality of electrical wires 515 extend the entire length of shaft 512. Shaft 512 includes a portion of the outer surface 508 adjacent the proximal end 516 that exposes the plurality of electrical wires 515. Exposing the plurality of electrical wires 515 allows shaft 512 to receive electrosurgical energy from housing 20 via cable 2, including electrical wires 28 that are connected to electrosurgical energy source, as described above. The distal end of the plurality of electrical wires 515 protrudes from the distal end 514 of shaft 512 facilitating the connection between the distal end 514 of shaft 512 to end effector assembly 100 (FIG. 1).

With reference to FIGS. 7 and 8, end effector assembly 100 of forceps 10 (FIG. 1) is shown. Each jaw member 110, 120 of end effector assembly 100 includes a jaw frame having a proximal flange portion 111, 121, an outer insulative jaw housing 112, 122 disposed about the distal portion (not explicitly shown) of each jaw members 110, 120, and a tissue-treating plate 114, 124, respectively. Proximal flange portions 111, 121 are pivotably coupled to one another about pivot 103 for moving jaw members 110, 120 between the spaced-apart and approximated positions, although other suitable mechanisms for pivoting jaw members 110, 120 relative to one another are also contemplated. The distal portions (not explicitly shown) of the jaw frames are configured to support jaw housings 112, 122, and tissue-treating plates 114, 124, respectively, thereon.

Outer insulative jaw housing 112, 122 of jaw members 110, 120 support and retain tissue-treating plates 114, 124 on respective jaw members 110, 120 in opposed relation relative to one another. Tissue-treating plates 114, 124 are formed from an electrically conductive material, e.g., for conducting electrosurgical energy therebetween for treating tissue, although tissue-treating plates 114, 124 may alternatively be configured to conduct any suitable energy, e.g., thermal, microwave, light, ultrasonic, etc., through tissue grasped therebetween for tissue treatment. As mentioned above, tissue-treating plates 114, 124 are coupled to the activation switch 4 (FIG. 1) and the energy source (not shown), e.g., via the electrical wires 28 extending from cable 2 (FIG. 1) through forceps 10 (FIG. 1), such that electrosurgical energy may be selectively supplied to tissue-treating plate 114 and/or tissue-treating plate 124 and conducted therebetween and through tissue disposed between jaw members 110, 120 to treat tissue.

The various embodiments disclosed herein may also be configured to work with robotic surgical systems and what is commonly referred to as “Telesurgery.” Such systems employ various robotic elements to assist the surgeon and allow remote operation (or partial remote operation) of surgical instrumentation. Various robotic arms, gears, cams, pulleys, electric and mechanical motors, etc. may be employed for this purpose and may be designed with a robotic surgical system to assist the surgeon during the course of an operation or treatment. Such robotic systems may include remotely steerable systems, automatically flexible surgical systems, remotely flexible surgical systems, remotely articulating surgical systems, wireless surgical systems, modular or selectively configurable remotely operated surgical systems, etc.

The robotic surgical systems may be employed with one or more consoles that are next to the operating theater or located in a remote location. In this instance, one team of surgeons or nurses may prep the patient for surgery and configure the robotic surgical system with one or more of the instruments disclosed herein while another surgeon (or group of surgeons) remotely control the instruments via robotic surgical system. As can be appreciated, a highly skilled surgeon may perform multiple operations in multiple locations without leaving his/her remote console which can be both economically advantageous and a benefit to the patient or a series of patients.

The robotic arms of the surgical system are typically coupled to a pair of master handle by a controller. The handles can be moved by the surgeon to produce a corresponding movement of the working ends of any type of surgical instrument (e.g., end effectors, graspers, knifes, scissors, etc.) which may complement the use of one or more of the embodiments described herein. The movement of the master handles may be sealed so that the working ends have a corresponding movement that is different, smaller or larger, than the movement performed by the operating hands of the surgeon. The scale factor or gearing ratio may be adjustable so that the operator can control the resolution of the working ends of the surgical instrument(s).

The master handles may include various sensors to provide feedback to the surgeon relating to various tissue parameters of conditions, e.g., tissue resistance due to manipulation, cutting or otherwise treating, pressure by the instrument onto the tissue, tissue temperature, tissue impedance, etc. As can be appreciated, such sensors provide the surgeon with enhanced tactile feedback simulating actual operating conditions. The master handles may also include a variety of different actuators for delicate tissue manipulation or treatment further enhancing the surgeon's ability to mimic actual operating conditions.

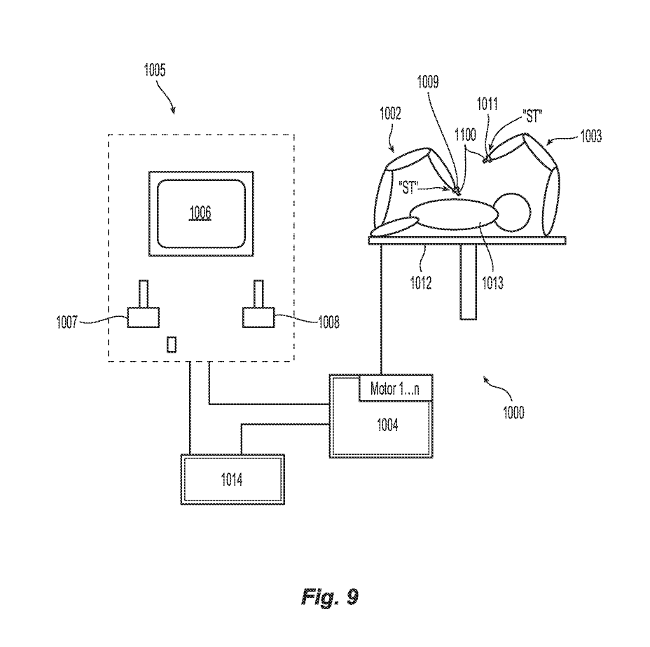

Referring to FIG. 9, a medical work station is shown generally as work station 1000 and may generally include a plurality of robotic arms 1002, 1003; a control device 1004; and an operating console 1005 coupled with control device 1004. Operating console 1005 may include a display device 1006, which may be set up in particular to display three-dimensional images; and manual input devices 1007, 1008, by means of which a person (not shown), for example a surgeon, may be able to telemanipulate robotic arms 1002, 1003 in a first operating mode.

Each of the robotic arms 1002, 1003 may include a plurality of members, which are connected through joints, and an attaching device 1009, 1011, to which may be attached, for example, a surgical tool “ST” supporting an end effector 1100, in accordance with any one of the embodiments disclosed hereinabove.

Robotic arms 1002, 1003 may be driven by electric drives (not shown) that are connected to control device 1004. Control device 1004 (e.g., a computer) may be set up to activate the drives, in particular by means of a computer program, in such a way that robotic arms 1002, 1003, their attaching devices 1009, 1011 and thus the surgical tool (including end effector 1100) execute a desired movement according to a movement defined by means of manual input devices 1007, 1008. Control device 1004 may also be set up in such a way that it regulates the movement of robotic arms 1002, 1003 and/or of the drives.

Medical work station 1000 may be configured for use on a patient 1013 lying on a patient table 1012 to be treated in a minimally invasive manner by means of end effector 1100. Medical work station 1000 may also include more than two robotic arms 1002, 1003, the additional robotic arms likewise being connected to control device 1004 and being telemanipulatable by means of operating console 1005. A medical instrument or surgical tool (including an end effector 1100) may also be attached to the additional robotic arm. Medical work station 1000 may include a database 1014, in particular coupled to with control device 1004, in which are stored, for example, pre-operative data from patient/living being 1013 and/or anatomical atlases.

From the foregoing and with reference to the various figure drawings, those skilled in the art will appreciate that certain modifications can also be made to the present disclosure without departing from the scope of the same. While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplification of particular embodiments. Those skilled in the art will envision other modifications within the scope and spirit of the claims appended hereto.

What is claimed is:

1. A surgical instrument, comprising:
a housing including at least one first electrical connector adapted to connect to an energy source;
a shaft extending distally from the housing, the shaft including a proximal end coupled to the housing and a distal end spaced-apart from the housing;
an end effector assembly disposed proximate the distal end of the shaft, the end effector assembly including at least one second electrical connector; and
a circuit board positioned on the shaft and extending from the proximal end of the shaft to the distal end of the shaft, the circuit board including at least one proximal contact at a proximal end thereof configured to electrically couple to the at least one first electrical connector and at least one distal contact at a distal end thereof configured to electrically couple to the at least one second electrical connector to thereby electrically couple the energy source to the end effector assembly.

2. The surgical instrument according to claim 1, wherein the circuit board is a flexible circuit board positioned on an interior surface of the shaft.

3. The surgical instrument according to claim 1, wherein the circuit board is formed on an interior surface of the shaft.

4. The surgical instrument according to claim 1, wherein the circuit board is formed on an exterior surface of a tube and the tube is disposed within the shaft.

5. The surgical instrument according to claim 1, further including a first jaw member and a second jaw member.

6. The surgical instrument according to claim 5, wherein the end effector assembly and the shaft each includes a sensor, each sensor is electrically coupled to a feedback circuit located on the circuit board.

7. The surgical instrument according to claim 6, wherein the feedback circuit calculates an angle of the first jaw member and second jaw member.

8. A method of manufacturing a surgical instrument, comprising:
positioning a circuit board on a shaft having a proximal end and a distal end such that the circuit board extends from the proximal end of the shaft to the distal end of the shaft, at least one proximal contact of the circuit board disposed at the proximal end of the shaft, and at least one distal contact of the circuit board disposed at the distal end of the shaft;
connecting at least one first electrical connector to the at least one proximal contact of the circuit board; and
connecting at least one second electrical connector to the at least one distal contact of the circuit board.

9. The method according to claim 8, wherein positioning the circuit board on the shaft includes aligning the at least one proximal contact with at least one proximal contact aperture defined through the shaft and aligning the at least one distal contact with at least one distal contact aperture defined through the shaft.

10. The method according to claim 8, further comprising connecting the proximal end of the shaft to a housing and connecting a distal end of the shaft to an end effector assembly.

11. The method according to claim 9, wherein positioning the circuit board on a shaft includes positioning a flexible circuit board on an interior surface of the shaft.

12. The method according to claim 8, wherein positioning the flexible circuit board to the interior surface of the shaft includes:
providing a flexible material;
forming a circuit board upon the flexible material;
positioning a proximal end of the circuit board to a proximal end of the shaft and a distal end of the circuit board to a distal end of the shaft; and
positioning the circuit board on the interior surface of the shaft.

13. The method according to claim 12, wherein positioning the circuit board on the shaft includes soldering.

14. The method according to claim 8, wherein positioning the circuit board on a shaft includes forming the circuit board on an exterior surface of a tube and disposing the tube within the shaft.

15. The method according to claim 14, wherein forming the circuit board including laser etching, physical etching, chemical etching or depositing.

16. The method according to claim 10, further comprising connecting an electrical wire within the housing to the shaft including:
connecting the shaft to a distal end of the housing;
connecting the electrical wire to a proximal end of the shaft; and
connecting the end effector assembly to a distal end of the shaft.

17. The method of according to claim 16, further comprising providing electrosurgical energy to the electrical wire.

18. A method of manufacturing a shaft of a surgical instrument, comprising:
forming a tube including a body defining an inner surface and an outer surface, and a central passageway extending longitudinally through the body; and
positioning a plurality of electrical wires through the outer surface of the body such that the plurality of electrical wires extends from a proximal end of the tube to a distal end of the tube.

19. The method according to claim 18, further comprising exposing a portion of at least one of the plurality of electrical wires.

20. The method according to claim 18, further comprising connecting a housing to the proximal end of the tube and connecting an end effector assembly to the distal end of the tube.
